(19) United States
(12) Patent Application Publication
(10) Pub. No.: US 2006/0150259 A1
(43) Pub. Date: Jul. 6, 2006

(54) Model of bone disease amenable to high-throughput screening

(76) Inventors: Paul Goldsmith, Cambridge (GB); Angeleen Louise Fleming, Cambridge (GB)

Correspondence Address: KLARQUIST SPARKMAN, LLP, 121 SW SALMON STREET, SUITE 1600, PORTLAND, OR, 97204, US

(21) Appl. No.: 10/544,001

(22) PCT Filed: Jan. 28, 2004
(86) PCT No.: PCT/GB04/00314

§ 371 Date: Jan. 6, 2006

(30) Foreign Application Priority Data
Jan. 28, 2003 (GB) 0301977.5

(51) Int. Cl. A61K 49/00; A01K 67/027

(52) U.S. Cl. 800/3; 800/20; 424/9.2

(57) ABSTRACT

Model fish and use thereof for screening for agents and genetic suppressors to treat bone disease or disorders, especially in zebrafish. Induction of a disease state and visualization of the skeleton in living zebrafish. Visualization of the skeleton in vivo facilitates screening for compounds that can be used in treatment of bone disease or genetic mutations that rescue or suppress the disease phenotype.

The present invention relates to a novel method for screening in a high-throughput fashion for treatments which alleviate osteoporosis and other bone and joint diseases or disorders, through the visualization of embryonic and larval skeleton of zebrafish exposed to an agent which induces osteoporosis.

Osteoporosis and other diseases of bone loss represent a major public health problem and there are an estimated 100 million people at risk of developing the disease worldwide. Osteoporosis is characterised by a gradual reduction in bone mass to a point where the skeleton is compromised, leading to bone fragility and susceptibility to fractures. The bisphosphonates, widely used in the treatment of osteoporosis, act by inhibiting bone resorption. However, there are few agents that promote or increase bone formation in patients who have suffered substantial bone loss, or for fracture healing. Recently, the statins have been proposed as agents that enhance osteoblast differentiation and bone formation in vitro, but there is conflicting data on the efficacy of statins in the treatment of osteoporosis in vivo.

Here we describe the invention of a zebrafish model of osteoporosis (OP) in which the disease shows pathological, biological and clinical relevance to the human condition. Furthermore, using techniques that enable rapid visualization of the skeleton in the living animal, we demonstrate that this model is amenable to screening for new anti-resorptive and anabolic treatments in high-throughput, as well as having applicability to osteoarthritis (OA) and fracture healing.

An additional major clinical problem is the side effects of steroid administration. Steroids are very effective at treating many human inflammatory diseases. However, the chronic nature of many inflammatory diseases, and the attendant side effects of long term steroid usage, pose a major clinical problem. In particular, steroid usage induces osteoporosis (Gulko and Mulloy, 1996). Patients taking steroids are commonly co-prescribed bisphosphonates, although their efficacy is only partial (Weinstein et al., 2002).

Glucocorticoid treatment causes a decrease in bone formation, by suppressing osteoblastogenesis, and an increase in bone resorption, by prolonging osteoclast survival (Weinstein et al., 1998; Weinstein et al., 2002). Glucocorticoid treatment is therefore an excellent experimental model for osteoporosis as it recapitulates both aspects of the human disease. By administering a glucocorticoid to the embryo medium at an appropriate dose and for an appropriate duration, in which are contained embryonic zebrafish an appropriate age, results in osteoporosis, as determined by detailed histological analysis.

We have discovered that glucocorticoid treatment can be used to induce OP in zebrafish larvae and demonstrated that the observed bone loss correlates with demineralization of the skeleton and an increase in osteoclast cell numbers.

Furthermore, since we are able to visualize and quantify changes in skeletal mineralisaton, this model is amenable to high-throughput screening for the identification of novel therapies for OP.

We have also invented a method for directly assaying for bone anabolism.

In order to screen for genetic suppressers of a disease it is desirable to be able to induce the disease state in a temporally controlled fashion to wild type embryos. This invention allows this. It is also important to be able to screen rapidly for the disease phenotype. This invention achieves this through the administration of a particular fluorescent dye to the embryo medium that marks bones. In particular, the bones of the head and axial skeleton are assayed in order for the results to be relevant to the human disease state.

This system is also amenable to high-throughput screening of therapeutic compounds. As an entire animal is being screened, optimal combinations of several possible anti-osteoporosis agents may be screened together.

Provided by the present invention are fish disease models which are not only representative of the underlying disease, but are also particularly amenable for use in subsequent screening. This allows in turn the identification of a human or other therapeutic.

The invention encompasses induction of bone disease in the model at any larval stage of the fish, and in adults.

The invention encompasses screening for anabolics at any larval stage of the fish, and in adults.

The invention is generally applicable to any of a variety of diseases and disorders, and a range of examples is specifically set out herein.

Since an aspect of the invention allows for visualisation of bones and the skeleton in fish, various models for study of bone and joint diseases are provided by the present invention, as discussed in the following paragraphs. Each of these models may be applied in methods of screening for compounds and/or genetic suppressors that treat (i.e. ameliorate at least a symptom of) such a disease.

Kyphosis—We have observed that aging zebrafish develop bony deformities akin to human kyposcoliosis. These changes are primarily observed in female zebrafish, indicating a hormonally driven effect. This therefore provides a powerful model of bony deformity in the aged.

Osteoarthritis (OA)—We have observed similarities between zebrafish and human joints and demonstrated that subcomponents of the joint can be visualized. This therefore provides a method for the screening for modifiers of subchondral bone biology and cartilage turnover applicable to the disease state of osteoarthritis.

Furthermore, we have observed differential effects of catabolic and anabolic compounds on endochondral and intramembranous bone. In the developing skeleton, bone is formed by one of two mechanisms, namely endochondral or intramembranous ossification. In the process of endochondral ossification, a cartilage template is first formed by chondroblasts and then becomes ossified as osteoblasts develop within the template. In the process of intramembranous ossification, the bone forms directly from osteoblasts within the mesenchyme. We have observed a difference in the bones affected by catabolic and anabolic agents. It appears that intramembranous bones are primarily affected when we induce OP with glucocorticoids. In contrast, we have observed that anabolic agents preferentially promote the development of endochondral bones. This latter observation may be of importance in developing compounds for OA, since the balance of chondroblasts and osteoblasts is critical in maintaining a normal joint. By assessing the activity and output of chondroblasts and osteoblasts in vivo, an understanding of the pathogenesis of joint diseases and possible treatments may be identified. For example, compounds that have an anabolic effect on bone formation (particularly on endochondral bones) may be detrimental in OA. Therefore, identifying agonists of certain receptors that have an anabolic effect in our bone assays (i.e. forming bone at the expense of cartilage), may promote cartilage formation and hence be relevant targets for the treatment of OA.

Immobilisation—By restricting the movement of zebrafish the rate of bone formation is reduced. Thus enforced movement restriction, or immobilization, offers a further method for modeling the environmental effects on bone biology.

Ovariectomy—The standard rodent models of osteoporosis involve ovariectomy. By administering a suitable compound, such as an anti-oestrogen, a similar model may be created in zebrafish via a chemical ovarectomy.

Bone strength assessment—Functional data on the strength of zebrafish bone may be gained through the use of microgauges.

Biochemical assessment—Biochemical measurements of bone breakdown products provide a further method for assessment of bone biology and OP. Samples may be conveniently taken from the fish water or from fish extracts.

Fin break—There is potentially a huge market for compounds that accelerate fracture repair, yet screening for such compounds has been limited in rodent studies since the severity of discomfort to the animals (fracture and restraint) is considered prohibitive to large scale screens. Fin break and repair in zebrafish may be analogous to fracture repair in mammals and this model may be useful for looking for factors that accelerate the repair process. The zebrafish fin consists of proximal cartilaginous rays and distal bony rays, each ray separated from the next by connective tissue. Each bony ray contains an arterial vessel and nerve and immunohistological analysis has demonstrated that scleroblasts (osteoblast-like cells) and osteoclasts are found in close association with these bony elements.

Following amputation, zebrafish fins are able to regenerate all of these components (Akimenko et al., 2003).

Furthermore, amputation of a section of the caudal fin is considered to be a very minor procedure, and is commonly used as a means for collecting DNA for genotype analysis. Amputation of a defined part of the caudal fin may produce a consistent and quantifiable repair, allowing for screening of agents (e.g. anabolic agents) that accelerate repair of the bone.

Thus, the present invention provides for screening for compounds and/or mutations that affect bone, cartilage and/or joint loss, other bone, joint and cartilage disorders, osteoarthritis, fracture healing, kyphoscolioss and other age-related bone changes.

The invention also provides for screening oestrogens and anti-oestrogens, next generation steroids with less bone effect and anabolic agents.

Analysis of the Head Skeleton

A third mechanism of bone formation is recognised in teleosts—that of acellular ossification. In this process, osteoid matrix that is laid down by cells on one surface and secreted outwards. As development proceeds, it is thought that this matrix then becomes populated by osteoblasts and osteoclasts. We have demonstrated that the vertebral column arises by a process of acellular ossification (Fleming et al., 2004) and hence is not a suitable tissue for the study of OP or anabolic agents in larval zebrafish, since is does not share the features of bone relevant to the disease in man. However, the head skeleton is formed by the processes of endochondral and intramembranous ossification and we have demonstrated the presence of osteoblasts, osteocytes and osteoclasts at larval stages. The zebrafish head skeleton is therefore ideal as a model to study OP and anabolic effects, since it contains many of the features relevant to OP and bone formation in man.

The present invention provides means, specifically a fish model as claimed and disclosed herein, and methods as claimed and disclosed.

The zebrafish is an organism which combines many of the advantages of mammalian and invertebrate model systems. It is a vertebrate and thus more relevant in models of human disease than Drosophila or other invertebrates, but unlike other vertebrate models it can be used to perform genetic screens.

A number of peer reviewed papers highlight and validate the use of zebrafish as a species in which to model human disorders. [Dooley and Zon (2000); Barut and Zon (2000); Fishman (2001).

The use of vertebrates offers the opportunity to perform sophisticated analyses to identify genes and processes involved in disease.

The inventors have appreciated that zebrafish offer the unique combination of invertebrate scalability and vertebrate modelling capabilities. They develop rapidly, with the basic body plan already having been laid out within 24 hours of fertilization. Moreover, their ex-utero development within a transparent capsule allows the easy in vivo visualisation of internal organs through a dissecting microscope. Many disease states can be modelled within the first week of life, at which time the embryos are only a few millimetres long and capable of living in 100 μl of fluid. This permits analysis of individual embryos in multi-channel format, such as 96 well plate format. This is particularly useful for drug screening, with many chemicals being arranged in 96 well plate format.

Alternatively, a population of fish in a petri dish or a tank may be employed. A population of fish may be treated together, and may be tested together, e.g. via addition of one or more or a combination of test substances to the water.

The zebrafish has a short maturation period of two to three months and is highly fecund, with a single pair of adults capable of producing 100 to 200 offspring per week. Both embryos and adults are small, embryos being a few mm and adults 2-3 cm long. They are cheap and easy to maintain. The ability to generate large numbers of offspring in a small place offers the potential of large scalability.

In addition to Zebrafish, other fish such as fugu, goldfish, medaka and giant rerio are amenable to manipulation, mutation and study, and use in aspects and embodiments of the present invention as disclosed herein.

In a further aspect, the present invention provides a method of making a fish model as disclosed, useful in or for use in a screen as disclosed herein and discussed further below.

In mutating a fish to determine the effect of such mutation on disease phenotype, a number of approaches may be taken.

Such a method may comprise providing a gene construct wherein a coding sequence of a disease gene is operably linked to a promoter that has the desired inducibility and/or tissue specificity, in the fish, introducing the gene construct into a fish embryo, causing or allowing the gene construct to integrate into the fish embryo genome, and growing the fish embryo into a viable fish.

A viable and reproductive fish may mate with one or more other fish, establishing a line of fish, e.g. zebrafish, transgenic for the gene construct comprising the disease gene operably linked to, and under regulatory control of, the promoter. A line of such fish, e.g. zebrafish, is useful in screens as disclosed.

In order to introduce a gene into a fish embryo, e.g. a zebrafish embryo, a gene construct is made, using techniques available to those skilled in the art. The construct may be released from a vector by restriction digest, and gel purified, for example by elution in 1×TE (pH8.0) and dilution to a working concentration of 50-100 ug/ml KCl containing a marker dye such as tetramethyl-rhodamine dextran (0.125%). Typically, 1 to 3 nl of this solution may be injected into single celled zebrafish embryos. Several thousand embryos may be injected.

Injected embryos are grown up and then mated with each other or to a non-transgenic wild-type fish. Transmission of the transgene to the subsequent generation is usually mosaic,, ranging from 2 to 90%. At least 100 offspring are typically analysed to establish whether the founder fish carriers the transgene.

Fish demonstrating a desired phenotype and/or genotype may be grown up and may be mated with wild-type fish. The parents and offspring may be matched and the offspring similarly assessed for phenotype and/or genotype. Those offspring with a particular phenotype, and hence likely germline transmission of an integrated disease gene construct, can be selectively bred. Some of the offspring may be sacrificed for more detailed analysis, e.g. to confirm the nature of the disease. This analysis may include in situ hybridisation studies using sense and anti-sense probes to the introduced gene to check for expression of the construct in cells of the fish, anatomical assessment such as with plastic sections to check for an effect on tissue or cells, and terminal deoxyuridine nucleotide end labelling (TUNEL) to check for apoptotic cell death in cells.

Families from which fish with the appropriate characteristics came may be maintained through subsequent generations. This maintenance then allows this new mutant strain to be entered into a secondary screen in accordance with further aspects of the invention.

A gene such as a disease gene sequence (e.g. heterologous to the fish e.g. zebrafish) to be employed in aspects and embodiments of the present invention may employ a wild-type gene or a mutant, variant or derivative sequence may be employed. The sequence may differ from wild-type by a change which is one or more of addition, insertion, deletion and substitution of one or more nucleotides of the sequence shown. Changes to a nucleotide sequence may result in an amino acid change at the protein level, or not, as determined by the genetic code.

Some aspects of the invention involve genetic rescue of an induced phenotype. Fish such as Zebrafish are particularly amenable to genetic rescue experiments.

Mutagens such as ethylnitrosourea (ENU) may be used to generate mutated lines for rescue screening, in either the F1-3 (for dominant) or F3 (for recessive) generations. (It is only by the third generation that recessive mutations can be bred to homozygosity.) ENU introduces point mutations with high efficiency, so any phenotype is most likely to be recessive. Retroviral vectors may be used for mutagenesis, and although they are an order of magnitude less effective than ENU they offer the advantage of rapid cloning of a mutated gene (see e.g. Golling et al.(2002) Nat Genet 31, 135-40. Mariner/Tc family transposable elements have been successfully mobilised in the zebrafish genome and may be used as mutagenic agents (Raz et al. (1998) Curr Biol 8, 82-8. ENU remains the most efficient and easy method available at the moment, and so is preferred for now.

Rescue strains are then created and the underlying genes mapped.

The mapping of mutant genes is comparatively easy. The density of markers on the fish genetic map, for example, is already considerably greater than that of the mouse map, despite the relatively recent popularity of zebrafish. Consult the harvard website on zebrafish, findable using any available web browser using terms “zebrafish” AND “harvard”, currently (28 Nov. 2002 and 22 Jan. 2004) found at (http://zebrafish.mgh.harvard.edu/mapping/ssr_map_index.htm 1), The Sanger Centre is sequencing the zebrafish genome with sequence currently (28 Nov. 2002 and 22 Jan. 2004) published at www.ensembl.org/Danio_rerio/. The site can be found using any web browser using terms “danio rerio” and “Sanger” or “ENSEMBL”. Around 70,000 ESTs have been identified and are being mapped on a radiation-hybrid map.

Another strategy for introducing effects, which may be random, on an aspect of behaviour or physiology in accordance with the present invention, is to down-regulate the function or activity of a gene, for instance employing a gene silencing or antisense technique, such as RNA interference or morpholinos. These can be either targeted against candidate genes, or generated against an array of genes as part of a systematic screen. It is relatively easy to inject RNA, DNA, chemicals, morpholinos or fluorescent markers into fish embryos, including zebrafish embryos, given their ex utero development.

A morpholino is a modified oligonucleotide containing A, C, G or T linked to a morpholine ring which protects against degradation and enhances stability. Antisense morpholinos bind to and inactivate RNAs and seem to work particularly well in zebrafish. Some disadvantages with this approach include the a priori need to know the gene sequence, the need to inject the chemical into the early embryo, potential toxic side effects and the relatively short duration of action. Additionally, they knock down the function of a gene, and thus do not offer the same repertoire of allele alterations as point mutations.

A further strategy for altering the function of a gene or protein as part of an in vivo screen, coupled to any of the various other components of the screening strategy disclosed herein, is to generate transgenic lines expressing protein aptamers, crossing these with the disease lines, or inducing disease by other means, then assaying for an altered disease state. Protein aptamers provide another route for drug discovery (Colas, 1996] but the ability to assay their effectiveness in vivo in accordance with the present invention markedly increasing their usefulness beyond in vitro screening methods.

A mutant fish such as a mutant zebrafish transgenic for a disease gene under control of a particular promoter and containing a mutation within a suppressor gene that lessens activity or effect of the disease gene on an aspect of behaviour or physiology of the animal is itself useful in a further assay for a test substance able to modulate or affect, preferably potentiate or increase the suppression effect of the suppressor gene. Clearly, the same applies where a mutation in a gene is identified that enhances or increases activity of a second gene.

Of course, the person skilled in the art will design any appropriate control experiments with which to compare results obtained in test assays.

In various further aspects, the present invention thus provides a pharmaceutical composition, medicament, drug or other composition comprising a suppressor gene or other gene or gene product or substance found to affect the disease gene of interest or suppression of the disease gene of interest, the use of such a material in a method of medical treatment, a method comprising administration of such a material to a patient, e.g. for treatment(which may include preventative treatment) of a medical condition, use of such a material in the manufacture of a composition, medicament or drug for administration for such a purpose, e.g. for treatment of a disorder, and a method of making a pharmaceutical composition comprising admixing such a material with a pharmaceutically acceptable excipient, vehicle or carrier, and optionally other ingredients.

One or more small molecules may be preferred therapeutics identified or obtained by means of the present invention. However, the invention may be used to identify appropriate targets for antibody mediated therapy, therapy mediated through gene targeting or protein targeting, or any of a variety of gene silencing techniques, including RNAi, antisense and morpholinos.

Alternatively, instead of or as well as an attempt to rescue the phenotype through the induction of genetic mutations, rescue may be achieved through application of a test substance, e.g. one or more chemicals. In this situation, all of the above methods need not require the mutation step for rescue (but will still require the mutation step if this is part of the procedure for induction of the disease state).

In a further aspect of the present invention, a fish in which one or more symptoms of a condition has been induced or is being modelled, may be treated with a test substance to screen for a substance capable of affecting the development of the condition. The effect of the test substance may be assessed by comparing an aspect of behaviour or physiology of treated fish with that aspect of behaviour or physiology of untreated fish to identify any treated fish with altered behaviour or physiology compared with an untreated fish, thereby to identify a test substance that affects development of the disease state.

The present invention provides means, specifically model fish for use in methods of screening for a test substance which when administered ameliorates symptoms of a disease state.

Fish may be treated with a test substance in a number of ways. For example, fish may be contacted with the test substance, it may be touched or rubbed on their surface or injected into them.

A further advantage of fish, especially zebrafish is the fact they live in water. This makes administration of test substances easy as they may be added to water in which the fish are. Zebrafish and other fish also readily absorb chemicals. The effective concentration of chemicals in the water often equates to the effective plasma concentration in mammals.

Different test substances may be added to each well of a multi-well plate, such as a 96 well plate, to identify that test substance exhibiting a beneficial or deleterious effect. There may be one or multiple fish in each well exposed to the test substance.

Moreover, the inventors have discovered that zebrafish are also DMSO (dimethyl sulphoxide) tolerant. This is important as DMSO is used as a solvent to dissolve many drugs. The inventors have established that zebrafish can tolerate 1% DMSO. Thus, a candidate drug or other test substance may be dissolved in DMSO and administered to zebrafish by adding to the fish water to give a final concentration of DMSO of at least up to 1%. This is employed in various preferred aspects and embodiments of the present invention.

The test substance may be added prior to the onset of the disease phenotype or concurrent with the onset of the disease phenotype. Preferably the test substance may be added subsequent to the onset of the disease phenotype.

The same test substance may be added to different wells at a different concentration. For example, test substance 1 may be added to well A1 at a concentration of 1 mM, to well A2 at a concentration of 100 uM, to well A3 at a concentration of 10 uM, to well A4 at a concentration of 1 uM and to well A5 at a concentration of 0.1 uM. Then test substance 2 to well B1 etc. The panel of test substances may be known drugs or new chemical entities.

Additionally, the test substances may be added in combination. For example, well A2 may contain test substance 1 and 2, well A3 test substance 1 and 3, well B2 test substance 2 and 3. Alternatively, every well may contain test substance x, with individual wells containing a panel of additional test substances.

In other options, a population of fish in a petri dish or a tank may be employed and treated together, e.g. via addition of one or more or a combination of test substances in the water.

Thus, zebrafish enable the entire biological pathway of a vertebrate to be screened in a high-throughput fashion.

The present invention in certain aspects and embodiments provides for screening for and preferably identifying or obtaining a substance that provides a synergistic combination with another substance, or for screening for and preferably identifying or obtaining two or more substances that together provide an additive or synergistic combination. Clinical benefit is often derived from synergistic combinations of drugs. Use of an in vivo system in accordance with the present invention allows for identification of such synergistic combinations.

Thus, in certain embodiments the invention comprises treating the fish, as discussed, with two or more substances, at least one of which is a test substance, and comparing the effect of the two or more substances in combination to determine the optimum effect (whether simultaneously or sequentially applied) on an aspect of behaviour or physiology with the effect of either or both of the two or more substances when applied individually or alone. Either all (or both) of the substances applied may each be a test substance, or one of the substances may be a drug known to have a beneficial effect in the disease that is the subject of the model, or at least an effect in the treated fish model.

The invention thus provides for screening for and preferably identifying or obtaining a substance that provides an additive effect to a known drug or a synergistic effect with the known drug. It also provides for screening for and preferably identifying or obtaining a combination of two or more substances that provide a synergistic effect, compared with the effect of the two substances when employed individually or alone.

Add-on therapies are useful because it is difficult to conduct clinical trials in which an existing drug is withdrawn from a patient and replaced with a new drug. The patient is deprived of a drug which has at least got some proven efficacy and some confidence in its side-effect profile. Additionally, the patient will be vulnerable to their disease during the phases of withdrawal of the existing drug and build up of the test drug.

In addition to a test substance, the fish may be a mutated animal rather than a wild-type animal. It is then possible to assay for interacting effects, either beneficial synergistic effects, or deleterious effects, of the mutation plus the test substances. Alternatively, the analysis may be of the known therapeutic agent and the genetic mutation to discover either a new drug target of benefit in combination with the known drug, or a genetic marker of use in predicting which patients are most likely to benefit (or not benefit) from prescription of the known drug.

In another embodiment, a combination of potential agents is administered to a fish having one or more symptoms of a disease, which may be generated as disclosed herein, to assess whether the combination is more effective than either of the individual agents.

For example, there are a variety of agents, either in clinical trials or currently prescribed. For the sake of this example, assume there are 11 drugs to be tested. It is possible that the various drugs act at different pinch points in biological pathways and that by judicious co-prescribing, an optimal combination may be found that is better than any drug alone, whilst with no worse a side effect profile. It would be very difficult to do clinical trials, or indeed mammalian studies to determine the optimum combination. The present invention allows this.

The present invention also provides for screening for and preferably identifying or obtaining a substance that ameliorates one or more side effects of an active substance, e.g. a therapeutically active substance. There are many drugs which have been discontinued in clinical trials, or are marketed but infrequently prescribed, not because they are not therapeutically effective, but because their side-effect profile is limiting. The side-effects may be relatively benign, or significant to the patient, such as renal damage (e.g. cyclosporin). It is desirable to allow the administration of such drugs, with proven beneficial effects, through the co-administration of an additional agent to improve the side-effect profile.

In accordance with the present invention, such agents are screened for in fish in which administration of the active substance induces a side-effect or other phenotype reflective or indicative of a side-effect. Thus in embodiments of the invention, an active agent is administered to fish having one or more symptoms of a disease and the side-effect of other phenotype is assessed for such animals when subjected to one or more test substances. This does not require a priori knowledge of action of the co-administered agent. In other embodiments, agents that achieve the desired therapeutic effect with a reduction of side-effects can be screened for and preferably identified or obtained by means of assessment of disease phenotype and side-effect phenotype. As with other aspects and embodiments of the present invention, this may involve co-administration of a primary compound together with either a battery of candidate substances, or together with randomly induced genetic mutation. With the latter approach, i.e. mutation, subsequent steps are needed to identify the appropriate co-therapeutic following identification of fish with a mutation that provides an ameliorative effect.

A diverse library of drug-like compounds, such as the LOPAC library (Sigma) may be used, or the Chembridge PHARMACOphore diverse combinatorial library. Other targeted libraries against particular targets classes may be used, such as ion channel libraries or G protein libraries.

Still further provided by the present invention is a method of identifying mutations, genotypes, allelic variations, haplotypes and genetic profiles associated with responsiveness to a therapeutic. There is an increasing move towards targeted prescribing, whereby the choice of therapeutic is influenced by genotyping the patient. Particular polymorphisms have been found to predict both the therapeutic effectiveness of a compound, and also the likelihood of suffering certain side effects. Such rationalised prescribing is cost-effective. It also makes clinical trials easier to run, as likely responders can be targeted, thus necessitating a smaller sample size to achieve statistical significance. However, for the moment, most drugs, both already prescribed or in development, do not have an appropriate test.

The present invention provides for assessing the effectiveness of various medications in combination with random genetic mutations to identify those mutations which either enhance or decrease the therapeutic-effectiveness and/or alter the side effect profile. This allows for identification of genes, polymorphisms, mutations, alleles and haplotypes associated with a particular response to a drug or other treatment, enabling development of appropriate genetic assays in humans to permit rationalised prescribing.

It is possible to introduce random mutations into the zebrafish genome, for example with the use of chemical mutagenesis (Solnica-Krezel et al., Genetics 1994, 136(4): 1401-20). It is also possible to make transgenic fish carrying exogenous genes.

In a further embodiment, rather than target the prescribing of a beneficial agent, or improve the efficacy of an already beneficial agent, the invention may be used to reduce the side effects of an agent which otherwise might not be prescribed because of its negative side effect profile. In this situation the deleterious side effect is assayed, with an improvement of this deleterious side effect being examined for through the result of an additional chemical or interactor gene.

It is well known that pharmaceutical research leading to the identification of a new drug may involve the screening of very large numbers of candidate substances, both before and even after a lead compound has been found. This is one factor which makes pharmaceutical research very expensive and time-consuming. Means for assisting in the screening process can have considerable commercial importance and utility. Such means for screening for substances potentially useful in treating or preventing a disorder or disease is provided by fish in accordance with the present invention. Modifier genes, such as enhancer or suppressor genes identified using the invention, and substances that affect activity of such suppressor genes represent an advance in the fight against disease since they provide basis for design and investigation of therapeutics for in vivo use, as do test substances able to affect activity or effect of a treatment, and substances that affect activity or effect of expression of a disease gene in a fish.

In various further aspects the present invention relates to screening and assay methods and means, and substances identified thereby.

Whatever the material used in a method of medical treatment of the present invention, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated.

Prescription of treatment, e.g. decisions on dosage etc, is within the responsibility of general practitioners and other medical doctors.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Examples of techniques and protocols mentioned above can be found in Remington's Pharmaceutical Sciences, 16th edition, Osol, A. (ed), 1980.

Vectors such as viral vectors have been used in the prior art to introduce nucleic acid into a wide variety of different target cells. Typically the vectors are exposed to the target cells so that transfection can take place in a sufficient proportion of the cells to provide a useful therapeutic or prophylactic effect from the expression of the desired peptide. The transfected nucleic acid may be permanently incorporated into the genome of each of the targeted cells, providing long lasting effect, or alternatively the treatment may have to be repeated periodically.

A variety of vectors, both viral vectors and plasmid vectors, are known in the art, see U.S. Pat. No. 5,252,479 and WO 93/07282. In particular, a number of viruses have been used as gene transfer vectors, including papovaviruses, such as SV40, vaccinia virus, herpesviruses, including HSV and EBV, and retroviruses. Many gene therapy protocols in the prior art have used disabled murine retroviruses.

As an alternative to the use of viral vectors in gene therapy other known methods of introducing nucleic acid into cells includes mechanical techniques such as microinjection, transfer mediated by liposomes and receptor-mediated DNA transfer, also administration of naked DNA or RNA, by simple administration, e.g. injection, of nucleic acid such as a plasmid, for instance to muscle.

The application of a test substance may then be as follows, in accordance with embodiments of the present invention:

1. A test substance is added to the fish either prior to the appearance of the disease state, at the time of induction of the disease state, or after the induction of the disease state. The first two situations are more likely to identify a prophylactic chemical, the latter a drug which reverts the disease state back to normal. The test substance may be a chemical and may be a random chemical administered in a high-throughput- fashion to fish in 96 well plate format, or a selected chemical administered to a clutch of fish in a Petri dish.

2. The fish is then screened for deviation from the initial disease state.

The following additional steps are highly desirable in screening, and their use is provided by the present invention in preferred embodiments:

Rather than add a single chemical, a combination of chemicals is added. For instance, a known therapeutic agent may be administered to all fish at a dose at which a further beneficial effect could still be detected. A random chemical library is then added to fish and an incremental effect screened for.

A further embodiment allows for detection of augmentation of a particular drug through a particular mutation, as follows:

1. Induce genetic mutation through any of the above.

2. Induce disease state.

3. Administer test chemical.

4. Assess whether the combination of the mutation plus chemical is greater than either alone.

5. The mutated gene is then used as a beneficial target, as described above.

A further embodiment of the invention allows identification of genetic factors which help determine the appropriateness of a particular therapeutic agent for a given patient. If the mutation augments the effect of the drug, that mutation is searched for in human homologues. Patients with this mutation should be preferentially prescribed the drug. If the mutation leads to a deleterious effect or lack of effect, then patients should avoid this drug.

A further embodiment of the invention allows identification of genetic or chemical factors which help prevent the side effects of an otherwise toxic drug. The following is an illustrative embodiment, and may be applied in other contexts for other diseases:

1. Drug X has a beneficial effect on disease Y, but causes side effect Z.

2. An zebrafish model is created which responds to treatment with drug X, but with the added complication of side effect Z.

3. The treated fish are co-treated with a panel of chemicals, (or alternatively are mutagenised as a route to a drug target).

4. Those fish which no longer show the side effect, but still show the beneficial effects are selected. The chemical is then used as a co-agent in patients to allow the safer administration of drug X, (or alternatively the mutagenised gene is mapped and used to develop the co-agent).

A further embodiment of the present invention involves attempting to modify the initial phenotype through a protein aptamer, rather than through a genetic mutation of chemical means. For example, a method may be performed in accordance with the following:

1. A construct coding for the desired aptamer (or random constructs for random aptamers) is injected into embryos to generate lines expressing the aptamer.

2. These lines are then crossed to the disease-expressing lines, or alternatively the disease state is induced in these lines.

3. The lines are then tested for deviation from the expected or intial phenotype.

4. If deviation occurs, the aptamer has in vivo proof of action and is used to derive a therapeutic agent.

Having identified fish with a mutation that confers rescue on a disease phenotype, the following steps may be performed:

1. The human homologue of the zebrafish rescue gene is cloned.

2. The same type of mutation is introduced into the human homologue.

3. The wild-type and mutated constructs are injected into the embryos.

4. The disease state is induced and assessed.

5. If the wild-type gene prevents the rescue, but the mutant gene retains it, this provides further evidence that the mutation is beneficial. However, a negative result does not necessarily rule out benefit.

6. The protein encoded by the human homologue is used for direct drug screens in vitro or directed in vivo screening.

The following is a description of one preferred method for the induction of disease and subsequent visualisation:

Induction of Osteoporosis with Prednisolone

Stock Solutions

Prednisolone stock (Sigma M0639) is made up as 50 ug/ml stock in embryo medium.

The stock is stored at 4° C. for a maximum of 2 months.

Toxicity

5 ug/ml prednisolone in E3M produces mild phenotype at 8-10 d.p.f. The embryos are viable beyond 10 d.p.f.

10 ug/ml prednisolone E3M produces readily scoreable phenotype at 8-10 d.p.f. Viable beyond 10 d.p.f. 20 ug/ml prednisolone in E3M produces strong phenotype at 8-10 d.p.f. but toxic in combination with rescuing drugs. For compound screening, a dose of 10 ug/ml prednisolone may be used, although other doses and durations of exposure may also be used.

Screening Protocol

1) Zebrafish larvae are immersed in embryo medium containing 10 ug/ml prednisolone from 3 d.p.f.

2) For in vivo examination of bone formation:
a) Saturated stock of Alizarin red S (Sigma A5533) in ddH₂O—stored at room temperature for up to 12 months.
b) 50 ul saturated alizarin red is added to OP-induced embryos in 10 ml E3M (i.e.0.5% alizarin red). They are stained for a minimum of 3 hours (up to 48 hours).
c) In vivo examination can be carried out from 8-10 d.p.f. For compound library screening, observations should be made at 10 d.p.f.
d) Staining is observed by viewing anaesthestised embryos under a fluorescence microscope using rhodamine filter.

3) For wholemount histological examination of bone formation:
a) Anaesthetise embryos at 10 d.p.f. by immersion in 0.2 mg/ml 3-amino benzoic acid ethylester (MS222) (Sigma).
b) Transfer embryos to glass vials. Remove all medium and replace with 4% paraformaldehyde (Sigma 44,124-4) in PBS.
c) Fix at room temperature for 4-24 hours. Stain as described below.
d) Staining is observed by viewing samples on a brighfield dissecting scope.

The phenotype following exposure to prednisolone is reduced staining in head skeleton and vertebrae.

The relevance of this model is demonstrated by the rescue of the disease phenotype with an agents known to rescue human IBD—a bisphosphonate.

Rescue of Osteoporosis with Bisphosphonates

Protocol:

1) Osteoporosis was induced with prednisolone as described above.

2) Etidronic acid (Sigma H6773) was made up as 10 ug/ml stock in embryo medium.

The stock was stored at 4° C. for maximum of 2 months.

3) Osteoporosis was rescued by co-administration of etidronic acid and prednisolone from 3 d.p.f. to 10 d.p.f.

4) Etidronic acid toxicity:

5 ug/mi etidronic acid—larvae viable beyond 10 d.p.f. when co-administered with 10 ug/ml prednisolone.

10 ug/ml etidronate—larvae viable beyond 10 d.p.f. when co-administered with 10 ug/ml prednisolone.

Wholemount Skeletal Staining

Stock Solutions

0.1% Alcian Green (Sigma A1182) in Acid/Alcohol Acid/Alcohol.—100ml 70% EtOH, 1 ml 37% HCl

Trypsin—concentration

1% KOH (Sigma P6310) in dH₂O

3% H₂O₂ (Sigma H1009) in 1% KOH—make weekly, store at 4° C.—in container with vented lid

Alizarin Red S (Sigma A5533)—saturated solution in dH₂O Glycerol (Sigma G7893)

Alcian Green for Cartilage

Staining Protocol:

1) Fix in 4% PFA (Sigma44,124-4) in PBS for minimum of 2 hours (maximum 24 hrs) at room temp

2) 3 washes in dH₂O for 10 mins each

3) Stain in 0.1% Alcian Green for minimum 2 hours (maximum overnight)

4) Differentiate in acid/alcohol for 30 mins (small larvae) to 24 hours (adults)

5) Rehydrate in dH₂O, replacing half the volume at a time (3 changes, 10 mins each)

6) Wash in dH₂O for 10 mins

7) Digest using trypsin—room temp for 10 mins (larvae), 37° C. overnight (adults)—duration judged by sight

8) Bleach in 3% H₂O₂ in 0.5% KOH until all pigment bleached

9) Rinse in 0.5% KOH

10) Gradually replace KOH with glycerol—equilibrated when embryos have sunk

11) Store in 100% glycerol with thymol

Results

Cartilage—blue, other tissues clear

Alcian Green and Alizarin Red for Cartilage and Bone

Staining Protocol:

1) Fix in 4% PFA in PBS for minimum of 2 hours (maximum 24 hrs) at room temp

2) 3 washes in dH₂O for 10 mins each

3) Stain in 0.1% Alcian Green for minimum 2 hours (maximum overnight)

4) Differentiate in acid/alcohol for 30 mins (small larvae) to 24 hours (adults)

N.B. Prolonged exposure to acid causes decalcification—keep times to minimum

5) Rehydrate in dH₂O, replacing half the volume at a time (3 changes, 10 mins each)

6) Wash in dH₂O for 10 mins

7) Digest using trypsin—room temp for 10 mins (larvae), 37° C. overnight (adults)—duration judged by sight

8) Bleach in 3% H₂O₂ in 1% KOH until all pigment bleached

9) Rinse in 1% KOH—3 washes, 10 mins-each

10) Stain in 1% KOH with enough alizarin red stock added to turn solution deep purple (2 hours for larvae, 8-24 hours for adults)

11) Differentiate in 0.5-1.0% KOH

N.B. Prolonged exposure to KOH causes decalcification—keep times to minimum

12) Gradually replace KOH with glycerol—equilibrated when embryos have sunk

13) Store in 100% glycerol with thymol

Results

Cartilage—blue, bone—red, other tissues clear

Alizarin Red for Bone

Staining Protocol:

1) Fix in 4% PFA in PBS for minimum of 2 hours (maximum 24 hrs) at room temp

2) 3 washes in dH₂O for 10 mins each

3) Digest using trypsin—room temp for 10 mins (larvae), 37° C. overnight (adults)—duration judged by sight

4) Bleach in 3% H₂O₂ in 1% KOH until all pigment bleached

5) Rinse in 1% KOH—3 washes, 10 mins each

6) Stain in 1% KOH with enough alizarin red stock added to turn solution deep purple (2 hours for larvae, 8-24 hours for adults)

7) Differentiate in 0.5-1.0% KOH

N.B. Prolonged exposure to KOH causes decalcification—keep times to minimum

8) Gradually replace KOH with glycerol—equilibrated when embryos have sunk

9) Store in 100% glycerol with thymol

Results

Bone—red, other tissues clear

In vivo stains for bone

Staining Protocol:

1) Craniofacial bones can be detected from 3 d.p.f, with good staining by 5 d.p.f. Vertebrae can be detected from 7 d.p.f. with good staining by 10 d.p.f.

2) Labelling agents are added to live larvae in E3M—minimum staining—2 hours, maximum staining—several weeks.

3) Any of the following fluorophores can be used:
Alizarin red (Sigma A5533)—50 ul saturated stock to 10 ml E3M—rhodamine filter
Quercetin (Sigma—Q0125)—forceps pinch to 10 ml E3M (not soluble)—FITC filter
Calcein (Sigma—C0875)—DAPI/FITC filter
Tetracycline—(Sigma—T3258)—DAPI/FITC filter Or any other fluorophore that binds to calcified or osteoid matrix.
4) Anaesthetise embryos and view using fluorescent dissecting scope with appropriate filter
   Material and Methods
   Maintenance of Stocks and Collection of Embryos

Fish were reared under standard conditions (Westerfield, 1995). Embryos were collected from natural spawnings, staged according to established criteria (Kimmel et al., 1995) and reared in embryo medium (5 mM NaCl, 0.17 mM KCl, 0.33 mMCaCl₂, 0.33 mM Mg₂SO₄, 10⁻⁵% Methylene Blue)

Induction of Osteoporosis

A stock solution of 50 ug/ml 6-α methyl prednisolone (Sigma) in embryo medium was used for the induction of OP. The stock solution was stored at 4° C. for maximum of 2 months. OP was induced by immersing zebrafish larvae from 3 days post-fertilisation (d.p.f.) in embryo medium containing 10 ug/ml 6-α methyl prednisolone (prednisolone). At 9 d.p.f. larvae were either labelled in vivo for demonstration of skeletal mineralisation or fixed and processed for wholemount skeletal staining.

Rescue of Osteoporosis with Etidronate

A 10 mg/ml stock solution of etidronate (Sigma) in embryo medium was used in all prednisolone rescue experiments. The pH of the solution was adjusted to pH 7.5 using NaOH. The stock was stored at 4° C. for a maximum of 2 months. The stock was stored at −20° C. for a maximum of 2 months. OP was induced by exposure of larvae to 10 ug/ml prednisolone from 3 d.p.f. to 6 d.p.f. At 6 d.p.f., medium containing 10 ug/ml prednisolone was removed and replaced with 10 ug/ml prednisolone in combination with etidronate. Etidronate was tested at a range of concentrations (1 to 15 μg/ml) to find the most effective rescuing dose. At 9 d.p.f. larvae were either labelled in vivo for demonstration of skeletal mineralisation or fixed and processed for wholemount skeletal staining.

Anabolic Bone Screens

A 1 mg/ml stock of parathyroid hormone (PTH) (Sigma) was made in 20 mM NaH₂PO₄ and 2.13g/L mannitol. A 100 mg/ml stock of cholecalciferol (Vitamin D)(Sigma) was made in dH₂O. All compounds were tested at a range of concentrations (PTH from 2.ng/ml to 500 ng/ml; cholecalciferol from 10 ng/ml to 1 μg/ml) to find the most effective anabolic dose. Larvae were reared from 3 d.p.f. to 8 d.p.f. in compounds with predicted anabolic bone effects then processed for skeletal staining as described.

Skeletal Staining

Embryos and larvae were fixed in 4% paraformaldehyde (PFA), then labelled with alizarin red as described herein. In vivo labelling was achieved by rearing fish in 0.05% alizarin red in embryo medium.

Quantification of Bone Mineralisation

Stained larvae were mounted on depression slides and visualised by fluorescence microscopy on a BX51 microscope (Olympus) and images were captured using a ColorView camera and AnalySis software (Olympus). The intensity of and area of alizarin red fluorescence was quantified for each treatment group, with a minimum of 5 samples per group, using colour threshold and area measurements in AnalySis (Olympus). Mean values and standard deviations were calculated using Excel software (Microsoft Office).

Antibody Labelling

10 d.p.f. larvae, previously reared in alizarin red, were fixed in 4% PFA and stained in wholemount as described (Westerfield, 1995) with minor modifications. The zns5 monoclonal antibody (University of Oregon) was used as a marker of osteblasts (1:200 dilution); TRAPC and cathepsin K (Novocastra) were used as osteoclast markers (1:600 and 1:40 dilutions respectively). Alexafluor 488 (Molecular Probes) was used as a secondary antibody. Antibody stained larvae were either viewed as wholemounts or sectioned. Larvae were embedded in O.C.T (Sakura) and frozen sections were cut and counterstained using Vectamount containing DAPI (Vector). Sections were viewed using a Leica TCS-NT confocal microscope. Alkaline phosphatase staining was performed on frozen sections using an ELF97 cytological labelling kit as described by the manufacturer (Molecular Probes) and visualised by fluorescence microscopy on an Axioplan 2 microscope (Zeiss). With a DAPI filter set the positive signal appears green.

Results

Zebrafish Model of Osteoporosis

The optical clarity, speed of development, and fecundity of zebrafish have made them a popular vertebrate model for the study of developmental biology and more recently as an animal model to study disease processes. This optical clarity allows the use of vital fluorescent dyes to mark discrete tissues and observe disease changes in the living animals and in post-mortem studies (e.g. labeling of bone as reported by Fleming et al., 2004).

We have discovered that we can use zebrafish, not only as a developmental model system, but also as the basis for modeling human bone and joint disease.

In vivo visualisation of the skeleton is achieved by the administration of a fluorescent dye to the embryo medium. Dyes that bind to calcified matrix can be used to label the entire skeleton (Du et al., 2001; Fleming et al., 2004). This not only provides a rapid method for assessing the skeleton but also allows measurement of the fluorescently labeled area or the fluorescent intensity of particular elements that can then be used to quantify bone size and density. Since the fluorescent dye is swallowed, labeling is also seen in the gut. Wholemount skeletal staining can also be performed on fixed tissue and can be used to generate a permanent record of changes in the skeleton following drug treatments.

Having optimized staining protocols for skeletal analysis in zebrafish larvae, we examined whether we could induce and detect osteoporosis in zebrafish larvae by administration of a glucocorticoid (prednisolone). In control samples at 9 d.p.f., the bony elements of head skeleton are well developed. When exposed to the prednisolone at 10 ug/ml, from 3 d.p.f. to 9 d.p.f., staining in the head skeleton is markedly reduced. When analysed using DIC optics, it is clear that bones are still present in these prednisolone treated samples, hence lack of staining indicates that the bone has become demineralised rather than that it has failed to develop. Using digital image analysis, we have measured the amount of stained mineralised tissue to quantify the OP-inducing effects of prednisolone at a range of doses. Samples were stained with alizarin red and the area of stained tissue was quantified with AnalySis software. The average stained area was calculated from five samples at each concentration. The amount of stained tissue decreased with increasing doses of prednisolone demonstrating that the degree of bone loss can be quantified. In subsequent studies, prednisolone has been used at 10 ug/ml.

Identification of Osteoblasts and Osteoclasts

One lack of obviousness feature of this model, related to our use of larval rather than adult fish, was that the action of prednisolone was to prevent bone formation, rather than to induce bone loss. We performed daily in vivo skeletal staining to confirm that bone is formed and lost following exposure to prednisolone suggesting that treatment with prednisolone increases bone resorption rather than blocking the development of bone. To further validate this model, we are able to quantify the relative numbers of osteoblasts and osteoclasts in control and OP samples. We have identified a number of suitable stains and antibodies that can be used to identify these cell types: alkaline phosphatase and tartrate-resistant alkaline phosphatase as an enzymatic markers of osteoblasts and osteoclasts, respectively; zns-5 as an antibody that marks osteoblasts (Johnson and Weston, 1994, Fleming et al., 2004); cathepsin K as an antibody that marks osteoclasts. Using digital image analysis, we can measure the number of stained cells in control and OP samples and hence quantify the number of osteoblasts and osteoclasts.

Contrary to previous studies that reported osteoclasts could not be detected in the zebrafish skeleton until 20 d.p.f.(Witten et al., 2001), we have demonstrated the presence of osteoclasts from at least as early as 5 d.p.f. and are more numerous in OP compared to control samples at 9 d.p.f..

Enzymatic detection of alkaline phosphatase, an osteoblast specific marker, was observed in the head skeleton at 10 d.p.f. (in a 10 um cryosection). Also performed was wholemount antibody stain of tartrate-resistant alkaline phosphatase (TRAP), an osteoclast specific marker. This was readily detected in the head skeleton. Calcified matrix was counter-stained with Alizarin red, osteoclasts were seen lying on the bone surface. Zns-5, a monoclonal antibody that marks osteoblasts, was observed in a confocal image of a parasagittal section through the head. Calcified matrix was counter-stained with Alizarin red, hence co-localisation of zns5 and mineralised matrix could be observed. Wholemount antibody detection of cathepsin K, an osteoblast specific marker, was also observed in the head skeleton. Individual osteoclasts were detected on the bone surface.

Rescue with Etidronate

Having established that exposure of zebrafish larvae to prednisolone results in biological and pathological changes relevant to those seen in humans with OP, we went on to examine the clinical relevance of this model by treating control and prednisolone-exposed fish with etidronate, a weak bisphosphonate, commonly used to treat OP in humans. A dose/response assay was performed with etidronate in the presence of prednisolone and the effects quantified by measuring fluorescence intensity in stained bones in vivo. Increasing doses of etidronate correlated with recovery from OP. This rescue is readily observed in wholemount skeletal preparations following treatment with etidronate at 10 ug/ml. This finding provides convincing evidence that such stains can be used to perform high throughput screens for compounds that prevent or reverse OP.

A dose/response study was performed using larvae exposed to varying doses of etidronate in the presence of prednisolone (at 10 ug/ml) from 3 d.p.f. to 9 d.p.f. Samples were stained with alizarin red and the area of stained tissue was quantified using AnalySis software. The average stained area was calculated from five samples at each concentration and plotted as a mineralization index.

Control samples were reared in embryo medium alone. There was a clear correlation between increasing doses of etidronate and rescue of the OP phenotype. Larvae were stained with alizarin red to visualise the mineralised skeleton. In control samples at 9 d.p.f., many features of the head skeleton were labelled. When exposed to prednisolone at 10 ug/ml, from 3 d.p.f. to 9 d.p.f., the amount of stained mineralised tissue was markedly reduced. When exposed to both prednisolone at 10 ug/ml and etidronate at 10 ug/ml, there was a clear increase in the amount of staining, hence rescue of the OP phenotype.

Anabolic Bone Screens

In addition to screening for the rescue of osteoporosis, we observed that anabolic bone effects could also be detected. We have adapted our screen to test compounds from 3 d.p.f. to 8 d.p.f., rather than 9 d.p.f., to look for anabolic effects, since increases in bone formation are more readily detected in younger animals. Cholecalciferol (Vitamin D) and parathyroid hormone (PTH)were tested for anabolic effects and showed a clear dose/response in relation to promoting bone. These findings validate our use of this model to screen for compounds with anabolic effects.

Selected Compound Screening

Studies on the action of PTH on bone mass have produced conflicting results (reviewed in Hruska et al., 1991). It has been demonstrated that PTH has an anabolic or OP-rescuing effect when given as a single high dose, but has a catabolic effect when given at a lower dose for a longer duration (Frolik et al., 2003). We have examined the role of PTH in our model both alone, as an anabolic agent and co-administered with prednisolone. We observe an anabolic effect at doses up to 100 ng/ml, but a catabolic effect at 206 ng/ml when administered continuously from 3 d.p.f to 9 d.p.f. However, when administered for 6-8 hours and then removed from the medium, we observe an anabolic effect at 200 ng/ml (Table 1). These findings are comparable with those from rodent studies. Optimisation of the anabolic screen with a single short administration may be useful in screening for compounds to accelerate fracture repair. Partial fin amputation or fin break and speed of repair may be used as an assay for compounds that accelerate fracture repair.

Discussion

Development of a Zebrafish Model of OP

While the rodent ovarectomy models of OP provide an excellent model system of the study of OP, such models are relatively slow to develop and measurement of the presence or severity of disease in the living animal are not easily measured. We have developed a zebrafish model of OP in which the disease is apparent within 1 week and that can be screened for increase in bone area or mass in a high-throughput fashion as we have developed rapid staining protocols that allow us to visualise the skeleton in both living animals and in fixed tissue. Moreover, the rate of disease induction and its severity is consistent both within and between assays since prednisolone is added to the embryo medium, ensuring equal exposure of all embryos to the same dose without repeated administration.

We observe pathological changes, measurable and quantifiable bone loss, and biological changes, increase in osteoclast cell number, in our disease model that are highly relevant to those seen in the human disease. In addition, we have validated our model using current therapies used to treat the human disease. We have demonstrated rescue of OP with bisphosphonates. Furthermore, we have demonstrated that the effect of such compounds is readily quantified in our assays, hence compounds can be ranked for efficacy.

Anabolic Effects—Acceleration of Bone Formation in a Developing System

During our observations of the prevention of OP with anabolic bone agents, we observed that such agent were able to promote bone formation above that of normal controls. This result was wholly unexpected, since our assay uses the developing larvae where is it largely assumed that the normal rate of development is maximal and cannot be pushed further. Our findings suggest that bone formation can be accelerated to yield significant increases within a treatment period of 5 days. This is due, in part, to the sensitivity of our assay system; since we are measuring small bones, very small increases become significant.

Additional Applications—Side Effect Profiling

OP is the unwanted side effect of a number of current and widely used therapies, including glucocorticoids and anti-oestrogens. Using zebrafish larvae as a rapid screening tool for effects on skeletal biology, next generation steroids and SERMs could be tested and quantification of changes in skeletal mineralisation used to rank the bone profiles on such compounds. In addition, we have developed a quantifiable zebrafish assay for screening novel anti-inflammatory therapies. The combination of such an anti-inflammatory screen with a ranking of OP side effects will provide a powerful tool for the dissocation of anti-inflammatory and OP effects in screens for next generation glucocorticoids.

TABLE 1
Summary of effects of increasing concentrations of
parathyroid hormone (PTH), with anabolic effects.
PTH produces anabolic effects when administered at low
doses (40 ng/ml) for the duration of the assay. At doses of
100 ng/ml and above, the effects of PTH are catabolic when
administered continually. However, brief administration of
200 ng/ml PTH on a single day results in increased bone
formation when assayed at 8 d.p.f.

| Dose of | Continuous or | | Summary of |
|---|---|---|---|
| PTH | brief exposure | Period of exposure | effects |
| 10 ng/ml | Continuous | 3 d.p.f. to 8 d.p.f | No change from |
| | | | controls |
| 20 ng/ml | Continuous | 3 d.p.f. to 8 d.p.f | No change from |
| | | | controls |
| 40 ng/ml | Continuous | 3 d.p.f. to 8 d.p.f | Anabolic effect |
| 100 ng/ml | Continuous | 3 d.p.f. to 8 d.p.f | Mild catabolic |
| | | | effect |
| 200 ng/ml | Continuous | 3 d.p.f. to 8 d.p.f | Strong catabolic |
| | | | effect |
| 200 ng/ml | Brief | 6 hours on 5 d.p.f, | Strong anabolic |
| | | effects noted on 8 d.p.f. | effect |

### REFERENCES

Akimenko et al. (2003). Developmental Dynamics (226): 190-201.
Barut and Zon (2000). Physiological Genomics (13): 49-51.
Colas et al. (1996). Nature (380): 548-550.
Dooley and Zon (2000). Current Opinions in Genetics and Development (10): 252-256.
Du et al. (2001). Developmental Biology (238): 239-246.
Fishman (2001). Science (294): 1290-1291.
Frolik et al. (2003). Bone (33):372-379.
Golling et al. (2002). Nature Genetics (31): 135-140.
Gulko and Mulloy (1996). Clin Exp Rheumatol. (2): 199-206.
Hruska et al. (1991). Contrib Nephrol. (91):38-42.
Johnson and Weston (1995). Genetics (141): 1583-1595.
Kimmel et al. (1995). Developmental Dynamics (203): 253-310.
Mundy et al. (1999). Science (286): 1946-1949.
Raz et al. (1998). Current Biology (8): 82-88.
Remington's Pharmaceutical Sciences (1980). 16th Edition, Osol, A. (ed). Mack Publishing Company, USA.
Solnica-Krezel et al. (1994). Genetics (136): 1401-1420.
Weinstein et al. (1998). Journal of Clinical Investigation (102): 274-282.
Weinstein et al. (2002). Journal of Clinical Investigation (109): 1041-1048.
Witten et al. (2001). Journal of Morphology (250): 197-207.
Westerfield (1995). The Zebrafish Book. University of Oregon Press. Eugene, Oreg.
van de Wyngaert et al. (2001). Acta Neurologica Belgica (101): 210-216.
Yaturu (2003). Endocr Pract. (4):315-20.

1. A fish model for bone disease, wherein a glucocorticoid is administered to medium containing embryonic fish to induce bone loss in the fish.

2. A fish model according to claim 1 wherein the glucocorticoid is prednisolone.

3. A fish model according to claim 1, wherein a fluorescent dye is administered to the medium to allow visualization of bones in the fish following staining by the dye.

4. A fish model for screening for disease phenotype, wherein a fluorescent dye is administered to medium containing embryonic fish to allow visualization of bones, cartilage and/or joints of the fish following staining by the dye.

5. A fish model according to claim 1, wherein the fish is a zebrafish.

6. A method of screening for a compound for use in treatment of a bone, cartilage and/or joint disease or disorder, comprising:
treating a fish model according to claim 1, with a compound,
identifying a compound that treats the disease or disorder.

7. A method according to claim 6 of screening for a compound for use in treatment of bone disease, comprising identifying a compound that decreases bone resorption or increases bone formation in the fish model, leading to improved bone mineralization.

8. A method according to claim 6, comprising high-throughput screening.

9. A method according to claim 6, comprising screening for optimal combinations of compounds that decrease bone resorption or increase bone formation in the fish model, leading to improved bone mineralization.

10. A method of screening for a genetic suppressor of a disease or disorder of bone, cartilage and/or joint, comprising:
identifying a genetic suppressor in a fish model according to claim 1 that suppresses the disease.

11. A method according to claim 10 of screening for a genetic suppressor of a disease of bone loss, comprising identifying a genetic suppressor of bone loss in the fish.

12. A method according to claim 6 comprising assessment of bone, cartilage and/or joints by means of X-rays, or a biochemical measure sampled from the fish water or from fish extracts.

13. A method of generating a fish model for bone disease, the method comprising administering a glucocorticoid to medium containing embryonic fish to induce bone loss in the fish.

14. A method according to claim 13 wherein the glucocorticoid is prednisolone.

15. A method according to claim 13, wherein a fluorescent dye is administered to the medium to allow visualization of bones in the fish following staining by the dye.

16. A method of generating a fish model for screening for disease phenotype, the method comprising administering a fluorescent dye to medium containing fish to allow visualization of bones of the fish following staining by the dye.

17. A method according to claims 11, wherein the fish is a zebrafish.

18. A fish model according to claim 4, wherein the fluorescent dye is administered to medium containing embryonic fish to allow visualization of bones, cartilage and/or joints of the fish following staining by the dye.

19. A method of generating a fish model according to claim 16, the method comprising administering the fluorescent dye to medium containing embryonic fish to allow visualization of bones of the fish following staining by the dye.
